SEQUENCE LISTING (12 sequences)

SEQ ID NO: 1 - DNA, 930 bp, Caulobacter crescentus
  CDS (1)..(930): HsfG
atgaacgcgcccgtcaacgagctgcgactcgagaacgccgcctgggccgcggcgcagccgcgcctgtcggtgctgatcccgaccttccgcgacgaccccagcgccctgctcaaggccctggaccacaccaatgccgccgtagaggtcgtggtgctggacgacggtggcggcgatgacgccctggccgaacggaccgcccgacggatcgagaagatgcgcaccccggcccgcttcgtgcgcctgtcgcgcaacgagggccgcgccaagggccgcaatcgcctggcctcgcacgcgcgcggcggccacttcctgttcttggacagcgacatgctgcccgacacccccgacttcctggaccgctggtcggcggtggcagataccggcgcggcggtagccttcggcggcttcaccctggaccagacccctcagcgtcccgagcacgccctgcaccgggccatggccctgaagagcgactgcacgcccgcgcccgagcgcgccaaggcgccggaaaagcacgtcttcacctcgaacctgctggttcgacgcgacgtcttcgagaccgtcggcttcgacgagggcttttccggctggggctgggaggatgtggagtgggcgatgcgcgtcgcacgccagcatccgatcctgcacatcgacaacaccgccacgcatctgggcctggaccccgccccggtgatggccgccaagtacgaacagtccgccgccaatttcgcgcgcgtggtggccagccaccgcgacgtggtcagcgcctatcccagctacaaggtcgccaagctgctgaaggcggttccgctcatcagcgtgtggcggccgcttctgaaacaggtggcgctggccgaggccgcgcccgtctccctccgcgccttcgccatgcggctctatcgcgcggcgctctacagcgaggccgtctga

SEQ ID NO: 2 - protein, 309 aa, Caulobacter crescentus
MNAPVNELRLENAAWAAAQPRLSVLIPTFRDDPSALLKALDHTNAAVEVVVLDDGGGDDALAERTARRIEKMRTPARFVRLSRNEGRAKGRNRLASHARGGHFLFLDSDMLPDTPDFLDRWSAVADTGAAVAFGGFTLDQTPQRPEHALHRAMALKSDCTPAPERAKAPEKHVFTSNLLVRRDVFETVGFDEGFSGWGWEDVEWAMRVARQHPILHIDNTATHLGLDPAPVMAAKYEQSAANFARVVASHRDVVSAYPSYKVAKLLKAVPLISVWRPLLKQVALAEAAPVSLRAFAMRLYRAALYSEAV

SEQ ID NO: 3 - DNA, 774 bp, Caulobacter crescentus
  CDS (1)..(774): HsfH
atgccgatggaattcgagaaggtcgacgcctacgagcccgaccgcagcctgaagggcaagctacgccgccggctgatccggctggcccaccgccggccggccaaggtggcgctggagcgtccgatggtgtcgttcagcttcgacgacgcccccgccaccgcctgcgaggccggcgccagggctctggaggcgcggggcctgcgcggcacctattacttcgcggcgggtctgacgggccgcgacggtccgatgggccgctacgccacgggcgaggacgcgcgccgcctgcatgaggccggccacgaaatcgcctgccacacctactcccacctcgattgtggccagtcgagccagaccgagacgctggcggatgtggaccgcaacgccgagagcctcgcggcctggggcgcgggagacccggtcagcttcgcctatccctacggggatgtggccgcgccggccaagaccgcgctgagcggccggttcaagacgctgcgcgcgctgcaccacgggctgatcaccgacggcgcggatctcaaccagacgcccgccgtcggcatcgagggcgaggatggcgagacagtcgccaaggcttggctcgacaaggccaaggcgcgcaaggcctggctgatcctctacacccacgatgtggctgggcagccgtcccaatggggctgcaccacggaggcgctggagcgcctgatcgatcgcgccctcgccgacgggttcgatgtggtgaccgtggccgagggctcgcggcggatcgggctctaa

SEQ ID NO: 4 - protein, 257 aa, Caulobacter crescentus
MPMEFEKVDAYEPDRSLKGKLRRRLIRLAHRRPAKVALERPMVSFSFDDAPATACEAGARALEARGLRGTYYFAAGLTGRDGPMGRYATGEDARRLHEAGHEIACHTYSHLDCGQSSQTETLADVDRNAESLAAWGAGDPVSFAYPYGDVAAPAKTALSGRFKTLRALHHGLITDGADLNQTPAVGIEGEDGETVAKAWLDKAKARKAWLILYTHDVAGQPSQWGCTTEALERLIDRALADGFDVVTVAEGSRRIGL

SEQ ID NO: 5 - DNA, 942 bp, Brevundimonas sp.
  CDS (1)..(942): HSF G
atgagcggctcccgcgccgatatccacgacaaccccggctgggccgccgcccggcccgaactgtcggtcctgatccccttcctgcgcgacgatcccgagcccctgctgcgccggctggataccgaaggccccgccctggccggccgggtcgagctgatcctgctggacgacggcacgggcgacgccgccctgaccgcccggctgaaggcggcgctggacgccctggccctgcccgcccgcctcgtcaccctgtccgccaatgaaggccgggcgcgcgggcgcaatcgcctgaccatcgccgcccgcgccggctcctggctgttcctcgacagcgacatgaagccggacagcccccgctttctccagacctgggtcgatctgattgcgcgcgacgaccccgccgtggccttcggcggtttctcgctgaaacaggcgccacgcgaccgccgcttcgccgtccaccgcgccctgtccggcgccagcgaatgcgtccgcgccgcccagcgcgccaggacgcccgagaaatacgtctacacctccaacctgctggtccggcgcgacgtcttcgccgtcgaatccttcgaccccggcttcaccggctgggggtgggaggatgtcgaatgggccatgcgggtcgcccgccggttcccggtggtccacatcgacaatccggccacccacatgggcctggacacggtgccggacctggcgcgcaagttcgaccagggcgccgccaacttcgcccgcgtggtcgccctgcaccccgagatcgtccagacctaccccagctacaaggccgcccgcgccctgaaacgcctgccgtccctgcccctgatccgtcgcctggcccggggcgccgccctgtcgccgctgttgccgacccgcacccgcgccttcgccctgcgtctgtatcgcgccgccgtctatgcggacgccatctga

SEQ ID NO: 6 - protein, 313 aa, Brevundimonas sp.
MSGSRADIHDNPGWAAARPELSVLIPFLRDDPEPLLRRLDTEGPALAGRVELILLDDGTGDAALTARLKAALDALALPARLVTLSANEGRARGRNRLTIAARAGSWLFLDSDMKPDSPRFLQTWVDLIARDDPAVAFGGFSLKQAPRDRRFAVHRALSGASECVRAAQRARTPEKYVYTSNLLVRRDVFAVESFDPGFTGWGWEDVEWAMRVARRFPVVHIDNPATHMGLDTVPDLARKFDQGAANFARVVALHPEIVQTYPSYKAARALKRLPSLPLIRRLARGAALSPLLPTRTRAFALRLYRAAVYADAI

SEQ ID NO: 7 - DNA, 918 bp, Asticcacaulis excentricus
  CDS (1)..(918): HSF G
atgaccgccaccgtcgtcaccaatgccgcccgtgaagccgcaccgccgccgcgcctgtcggtgctgatccccttctataaggaaagcccggccaccctgctgcgcgccctgacgccgacaccgggggtggaaatcgtgctgctcgatgacgggtccgggcaggccgagctgacgcaggaggtgatgtcaacgattgatgaagtcgccctgcccgccaccttcctcagcctgacgcacaatgaaggccgggcgcgtgggcgcaaccgcctgacgcaagccgcgcgcggcgactatttcctgtgcctcgattccgacatgctgcctgatgcgccggacttcctgacgcgctggcttgaggtgatggacgacaatcctgccgtagtgtttggcggcttttcgctgcttcaggccccgatggacaaacgctttgccatccaccgcatgatggcagccaaaagcgactgcctcaatgccgccacccgcgccctgatgtccgagaaatacgtctttacctcgaaccttcttatccgccgcgacgtctttgcgacgcaggatttcgacagcagcttcaccggctggggctgggaagacgtcgaatgggctatgcgcgtggcgcaacagtttggcgtgcagcatatcgacaaccccgccacccatctgggcctcgacacggcggacacgctgatgcgcaagtatgagcaatcggtcggcaacttcgcccgcgtcgttgacaaacacccgcaggtcgtcgccacctatgccagctaccgcgcggcgcgcctgatccgcaaattgcccctgcgcggggccctacgcgccaccttgcgcggcgtgatccgcaacgaagccctgccgctgaaggcccgcgcctttggcctgcgcctgtaccgcgcggccctttattctgaggtcgtctga

SEQ ID NO: 8 - protein, 305 aa, Asticcacaulis excentricus
MTATVVTNAAREAAPPPRLSVLIPFYKESPATLLRALTPTPGVEIVLLDDGSGQAELTQEVMSTIDEVALPATFLSLTHNEGRARGRNRLTQAARGDYFLCLDSDMLPDAPDFLTRWLEVMDDNPAVVFGGFSLLQAPMDKRFAIHRMMAAKSDCLNAATRALMSEKYVFTSNLLIRRDVFATQDFDSSFTGWGWEDVEWAMRVAQQFGVQHIDNPATHLGLDTADTLMRKYEQSVGNFARVVDKHPQVVATYASYRAARLIRKLPLRGALRATLRGVIRNEALPLKARAFGLRLYRAALYSEVV

SEQ ID NO: 9 - DNA, 768 bp, Asticcacaulis excentricus
  CDS (1)..(768): HSF H
atgaactatgccgaagcctatgctgccgatcgttcgctgtatggcaagctgcgccgccgcgcctcaaagctgctgttccgcaaaccggcgcagttgaaagggctgacgaaaccgctgctgaccttttcgtttgatgatgcccctcagtcggcggccattgcaggggcgggcatactggaacgccacggctatcgcgccacctatttcatctccgccggactgatggggcaggacagccatttcggcccctacaccaccgccgctcagatcgccgccctgaatgcgcgcggccatgagatcgcttgccacaccctgatgcacatcgactgtggtcaggcgaagggggcagacattgctttaagcgtggacaaaaatcaaaagcttattcaatcacttggcatgcccacttcaaccacttttgcctacccttatggcgatgtatcaccacaggccaaggcggtgctgggcgaccgctatcgctcctcgcgtgccctgcaccacggtctgatccgctccggcagcgacctcaatcaggcccccgccatcggcatcgaaggcgacgccggccaacggctggcgctggagtggatggcacaggcgctcaaaaccccgcaaagctggttggtactctacacccacgatgtgcgtgaaaatccctcgccctggggctgcacgccggagactctggagacgctggccaccgccgcccagactctggggttcgaggtcgtcacctatgccgaaggggcgcgccgcgcgggcggctaa

SEQ ID NO: 10 - protein, 255 aa, Asticcacaulis excentricus
MNYAEAYAADRSLYGKLRRRASKLLFRKPAQLKGLTKPLLTFSFDDAPQSAAIAGAGILERHGYRATYFISAGLMGQDSHFGPYTTAAQIAALNARGHEIACHTLMHIDCGQAKGADIALSVDKNQKLIQSLGMPTSTTFAYPYGDVSPQAKAVLGDRYRSSRALHHGLIRSGSDLNQAPAIGIEGDAGQRLALEWMAQALKTPQSWLVLYTHDVRENPSPWGCTPETLETLATAAQTLGFEVVTYAEGARRAGG

SEQ ID NO: 11 - DNA, 774 bp, Caulobacter sp.
  CDS (1)..(774): HSF H
atgcagacttcattcgaaaaggtcgacgcctacgagcccgaccgcaccctgaagggcaagctgcggcgccggctgatccgcctggcccatcgccgtccggcccgggtgaagctggaacggccgatggtgtcgttcagcttcgacgacgccccggccacggcctgcgaggccggagcccgggtgctggagcaacgcggcctgcgcggcacctactatttcgccgctggcctgaccgggcgcgacggaccgatgggccgtttcgccaccggcgaggacgccgcccgcctgcacgccgccggccacgagatcgcctgccataccttctcgcacctcgactgcggccaggcctcgcgcgccgagaccctggccgacgtcgaccgcaacggcgcggccctggcctcgtggggcgtcggcgagccggtcagcttcgcctacccttacggcgacgtcgccagcccggccaagaccgccctggccggccgcttcaagaccctgcgcgccctgcaccatggcctgatcgccaacggcgccgacctcaaccaggccccggcggtcggcatcgagggcgaagacggcgagcaggtcgccctggactggctggacaaggcccacgggcgcaacgcctggctgatcctctacacgcacgacgtctcgctcgagccgtcgcaatggggctgcacgacgggagcgctggagcggttggtcgacggcgcgctgctggcggggttcgatgtggtgaccgtggccgaaggggcgcagcggatcgggctgtag

SEQ ID NO: 12 - protein, 257 aa, Caulobacter sp.
MQTSFEKVDAYEPDRTLKGKLRRRLIRLAHRRPARVKLERPMVSFSFDDAPATACEAGARVLEQRGLRGTYYFAAGLTGRDGPMGRFATGEDAARLHAAGHEIACHTFSHLDCGQASRAETLADVDRNGAALASWGVGEPVSFAYPYGDVASPAKTALAGRFKTLRALHHGLIANGADLNQAPAVGIEGEDGEQVALDWLDKAHGRNAWLILYTHDVSLEPSQWGCTTGALERLVDGALLAGFDVVTVAEGAQRIGL

(19) United States
(12) Patent Application Publication
(10) Pub. No.: US 2011/0284161 A1
(43) Pub. Date: Nov. 24, 2011

(54) BIOLOGICAL GLUE AND METHOD FOR OBTAINING A BIOLOGICAL GLUE

(75) Inventors: Laurence VAN MELDEREN, Waterloo (BE); Gilles Vanwalleghem, Bruxelles (BE); Pierre-Alexandre Drèze, Huy (BE); Marie Machiels, Bruxelles (BE); Aline Gheeraert, Bruxelles (BE); Amandine Caprasse, La Hulpe (BE); Hugo Herter, Bruxelles (BE); Laetitia Warny, Waterloo (BE); Eric Karengera, Bruxelles (BE); Quentin Vanhaelen, Moulbaix (BE); Calvin Cambier, Bruxelles (BE); Alexis Nsamzinshuti, Denderleeuw (BE)

(73) Assignee: Universite Libre de Bruxelles, Bruxelles (BE)

(21) Appl. No.: 13/086,154

(22) Filed: Apr. 13, 2011

Related U.S. Application Data
(60) Provisional application No. 61/346,168, filed on May 19, 2010.

(51) Int. Cl. C12P 1/04; C12N 15/63; B32B 37/14; B32B 37/02; B32B 37/12; C12P 1/00; C12N 1/21

(52) U.S. Cl. 156/336; 435/170; 435/41; 435/320.1; 435/252.33

(57) ABSTRACT

A method obtains a biological glue from a bacteria.

This application claims benefit of Ser. No. 61/346,168, filed 19 May 2010 in the United States of America and which application is incorporated herein by reference. A claim of priority to the extent appropriate is made.

### FIELD OF THE INVENTION

The present invention is in the field of genetic engineering and is related to a new biological glue that is obtainable from a genetically modified cell, preferably from a genetically modified Escherichia coli. The present invention concerns also the method for obtaining this biological glue and the use of this biological glue for fixing solid elements.

### BACKGROUND OF THE INVENTION AND STATE OF THE ART

Glues are used to bind two items together and almost every solid item can be fixed by glues.

Glues comprising synthetic adhesives can be toxic and furthermore are mixed with solvents that need to evaporate to obtain a hardening of the adhesive. These volatile organic compounds, intrinsic components of today's glues may pollute the environment and are often toxic.

Glues made of natural adhesives (e.g. comprising proteins and/or saccharides) are currently less used, probably due to their price, to their lower flexibility of use and to their reduced adhesivity.

The international patent application WO90/04963 discloses the development of bio-adhesive based on material (mainly proteins; including a short peptide of 14 amino acids) from fimbriae of Escherichia coli. However, either this product needs to be obtained after a long purification process resulting into a substantially pure peptide or the peptide needs to be chemically synthetized. In every case, the required process is complex and time-consuming.

Toh et al. (J. Bacteriol. 2008 190(21):7219-7231) disclose an holdfast system, which is based upon an elastic material rich in N-acetylglucosamine that is strongly adhesive and they further disclose genes that are essential for the synthesis of this system in Caulobacter crescentus.

Therefore, there is a need to develop glues and a method for obtaining it that do not present the drawbacks of the state of the art and that combine all the advantages of a natural glue being low toxic, degradable and efficient, having a broad spectrum of uses and being obtainable by a simple and cheap industrial process.

### SUMMARY OF THE INVENTION

The inventors surprisingly found a method to obtain a glue from a bacteria, preferably a Gram-negative bacteria, such as Escherichia coli.

A first aspect of the invention is related to a method to obtain a (biological) glue by a cell and comprising the steps of:
selecting a cell, preferably a prokaryote cell, lacking of hfsG and hfsH genes (nucleotide sequences encoding the corresponding hfsG and hfsH proteins or portion(s) thereof presenting at least 50%, 60%, 70%, 80% or at least 90% of the enzymatic activity of the corresponding wild type proteins);
introducing these (exogenous) hfsG and hfsH genes and possibly one or more genes involved for the production of one or more natural pigments, into this cell, preferably these genes being carried by an extrachromosomal replicon, such as a plasmid, to express the corresponding proteins (or portions thereof) encoded by these exogenous) hfsG and hfsH genes (nucleotide sequences); and
recovering the biological glue from the cell (following expression of these proteins or their portions).

By hfsG gene, it is preferably meant a gene (or nucleotide sequence) encoding a glycosyl transferase and more preferably a gene (or nucleotide sequence) encoding a protein having at least 30%, 40%, 50%, 60%, 70%, 75%, 80%, 85%, 90%, 95%, 96%, 97%, 98%, 99% and even 100% sequence identity with SEQ.ID.NO.2, SEQ.ID.NO.6 and/or SEQ.ID.NO.8 (including homologous sequences that may exist in other species). An hfsG gene (or nucleotide sequence) means also the full length sequence SEQ.ID.NO.2 SEQ.ID.NO.6 and/or SEQ.ID.NO.8 or a portion thereof encoding a peptide having at least 80%, more preferably at least 90% of the enzymatic activity of the wild-type enzyme (encoded by the nucleotide sequence SEQ.ID.NO.1, SEQ.ID.NO.5 and/or SEQ.ID.NO.7).

By hfsH gene, it is preferably meant a gene (or nucleotide sequence) encoding a polysaccharide deacetylase and more preferably a gene (or nucleotide sequence) encoding a protein having at least 30%, 40%, 50%, 60%, 65%, 70%, 75%, 80%, 85%, 90%, 95%, 96%, 97%, 98%, 99% and even 100% % sequence identity with SEQ.ID.NO.4 SEQ.ID.NO.10 and/or SEQ.ID.NO.12. An hfsH gene means also the full length sequence SEQ.ID.NO.4 SEQ.ID.NO.10 and/or SEQ.ID.NO.12 or a portion thereof encoding a peptide having at least 80%, more preferably at least 90% of the enzymatic activity of the wild type enzyme (encoded by the sequence SEQ.ID.NO.3 SEQ.ID.NO.9 and/or SEQ.ID.NO.11).

Preferably, the biological glue is obtained by drying the culture medium in which the glue producing cell has grown, more preferably medium conditioned by this cell or of the lysis of the cell.

Another aspect of the present invention is related to a recombinant E. coli cell having further incorporated an exogenous genetic construct (possibly carried by an extrachromosomal replicon) carrying hfsG and hfsH genes (nucleotides sequences) and possibly additional expression elements allowing their maintenance and expression, such as an origin of replication and/or promoter/operator sequence(s) and possibly one or more genes (nucleotide sequences encoding enzymes and others proteins) involved in the synthesis of natural pigments by these cells (Carotenoids, Melanin and/or Violacein) such as CrtE, CrtB, CrtI and possibly CrtY, preferably from Pantoea ananatis, MeI A gene, preferably from Rhizobium etli and/or Vio A-E genes, preferably from Chromobacterium Voilaceum ATCC 12472), so as to obtain the production of a pigmented (colored) biological glue.

Advantageously, the cell of the invention may also further comprise a gene (nucleotide sequence) encoding a toxic (poison) protein or molecule, such as a gene (nucleotide sequence) encoding a bacteriocin or preferably the ccdB gene (nucleotide sequence) in its genome (chromosome), but no corresponding antidote gene (nucleotide sequence) of this toxic (poison) protein (being preferably ccdA) present in the genome (chromosome) of the cell. However, this corresponding antidote gene (nucleotide sequence) is possibly present on an extrachromosomal replicon, such as a plasmid comprising these exogenous hfsG and hfsH genes to allow their maintenance (stabilisation) in the cell.

Alternatively, or in addition, the (prokaryote) cell of the invention (possibly used in the method of the present invention) further comprises genes (nucleotide sequences) encoding proteins involved in the secretion of polysaccharides, such as HfsA, HfsB and HfsD proteins (of Cauobacter crescentus) or homologs thereof.

Preferably, the (prokaryote) cell of the invention (possibly used in the method of the present invention) further comprises genes (nucleotide sequences) encoding HfsA and/or HfsB and/or hfsD proteins.

Advantageously, all the genes inserted into the (prokaryote) cell of the present invention are under the control of inducible promoters.

The most preferred prokaryote cell is Escherichia coli.

This prokaryote (E. coli) comprises thus genes encoding HsfG and HsfH and preferably genes (of Cauobacter crescentus) encoding HsfA and/or HsfB and/or HsfC and/or HsfD and/or HsfE and/or HsfF proteins.

Advantageously, this prokaryote (E. coli) has further been modified by the deletion of the cps and/or the cellulose and/or the PGA operon(s).

Another aspect of the present invention is a polysaccharidic glue, preferably from prokaryotic origin, being not Caulobacter crescentus and being obtainable, preferably being obtained by the method of the invention.

A last aspect of the present invention is a (biological) polysaccharidic glue preferably from prokaryotic origin enriched in (non acetylated) hexosamine.

Preferably, the (biological) polysaccaridic glue of the present invention is water-sensitive.

By “enriched in (non acetylated) hexosamine”, it is meant that the polysaccharide present in the glue comprise at least 1%, preferably at least 5%, more preferably at least 10%, still more preferably at least 15% and even possibly at least 20% of (non acetylated) hexosamine, the percentages being given on a molar basis (appreciatively equal to a weight basis).

By polysaccharide, it is meant a polymer made of sugar (saccharide) monomers (including sugar (Saccharide) monomers derivatives).

Advantageously, the (biological) glue of the present invention comprises at least 50% (w:w) of polysaccharides and/or may contain one or more natural pigment(s), preferably selected from the group consisting of carotenoids, melanin, violacein or mixture thereof.

More precisely, it is meant that at least 10, preferably at least 20, more preferably at least 50 sugar monomers comprise one single polysaccharide.

Preferably, the biological glue of the present invention further comprise other molecules from prokaryotic origin, preferably selected from the group consisting of proteins, proteoglycans, D-amino acids and CpG-rich DNA sequences, being more preferably D-amino acids such as D-Ala and D-Isoglutamate.

Another aspect of the invention is related to a matrix comprising the cell of the invention and nutrients (and support elements to form this matrix, preferably in the form of a gel such as an aqueous medium, saccharides and/or proteins) for the glue synthesis by this cell and to a method which may comprise the use of this matrix for a fixing of different solid elements by the (possibly pigmented (colored)) biological glue obtained from this cell, and which comprises the step of introducing between these solid elements the matrix of the invention for a sufficient time until when the nutrients present in the matrix are consumed by this cell leading to its lysis and to the release of the produced biological glue by the cell and to the fixing of the solid elements (together) by the released glue.

The present invention will be described hereafter in the detailed description of the invention in references to the enclosed figures and example presented as a non limiting illustration of the present invention.

### BRIEF DESCRIPTION OF THE FIGURES AND TABLES

FIG. 1 is a schematic view of one of the genetic construction developed and used.

FIG. 2 represents the strong adhesion of glass beads to a plastic medium obtained by the use of glue of the invention.

### DETAILED DESCRIPTION OF THE INVENTION

Caulobacter crescentus is a gram-negative bacterium studied for the properties of its cell cycle. This bacterium is widely spread in aquatic environments and exists either as a motile or as a sessile cell. In the sessile stage, C. crescentus sticks to surfaces by synthesizing adhesive including a complex holdfast structure.

The hfsE, hfsF, hfsG and hfsH are organized in operon and their products are required for the minimum repeat unit holdfast synthesis, while the polymerization of the repeat unit of the holdfast substance needs the expression of hfsC and hfsI genes.

In addition, the products of 3 adjacent hfsD, hfsA and hfsB genes are involved in the holdfast export.

By introducing some of these genes (at least hfsG and hfsH) into another prokaryote, such as E. coli, the inventors surprisingly obtained a mass-production of a biological glue.

### Example 1

The inventors selected competent E. coli cells purchased from Delphigenetics (www.delphigenetics.com) and transformed them using a plasmid encoding both hfsG and hfsH genes under the control of a lactose-inducible promoter and further bearing genes encoding for resistance to ampicilin and kanamycin as selection-pressure. The plasmid furthermore bears a reporter gene being RFP, as an indicator of HfsG and HfsH expression.

The inventors further developed a more robust system by placing the expression of 4 genes under the control of a first repressor (c2 P22) and of a promoter (Lux). These genes are luxI, luxR, hfsG and hfsH genes. At basal conditions, c2 P22 is expressed, and represses the expression of these 4 genes (no Lux is expressed).

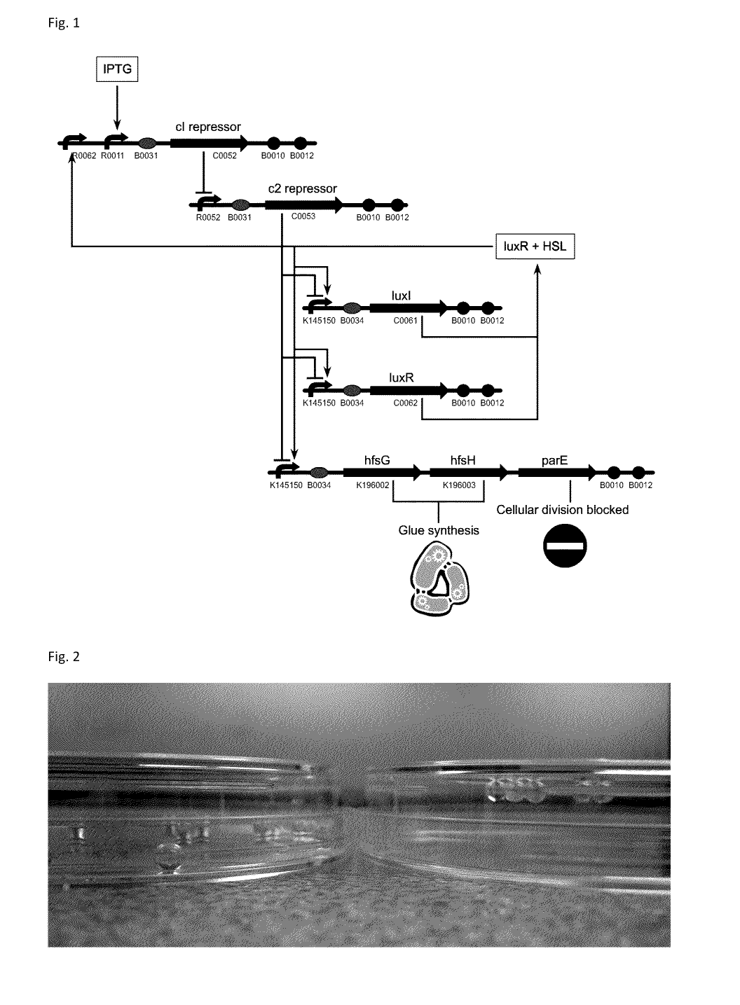

In this system, c2 P22 is placed under the constitutive ‘hydrid’ cI434 promoter (coding for a repressor from phage 434), whose activity is controlled by IPTG and by LuxR/HSL. When IPTG is added (for instance 1 mM) to the medium, cI434 repressor is produced, blocking the activity of the c2 P22 repressor. Therefore, the inhibition of expression of LuxI, luxR, and hfsG and hfsH genes by c2 P22 is released by IPTG. In addition to the classical expression system triggered by IPTG, the expression at a basal level of LuxI and LuxR (LuxR/HSL complex) then fully activates cI434 expression and therefore increases the expression of hfsG and hfsH genes, but also of LuxI and luxR, resulting in a robust and stable expression of the target genes hfsG and hfsH (FIG. 1).

The inventors grow the transformed E. coli cells in liquid medium. The transformed bacteria colonies showed a filamentous aspect. Thereafter, glass beads were added to the culture in a Petri dish and, after 7 h of incubation, the inventors observed a perfect adherence of the glass beads to a plastic Petri dish containing the (dried) medium of culture of the E. coli cells of the invention. The inventors furthermore observed the resistance of this adherence to physical perturbations, reflecting an unexpected strength of this biological glue (FIG. 2).

The inventors further noticed that water negatively impact the adhesive effect of the glue they developed, that was surprising, since C. crescentus live and adhere to solids in an aqueous environment. Therefore, the inventors conclude that the glue they obtained is different of the adhesion system of C. crescentus.

The inventors further noticed that a significant part of the glue is retained in inclusion bodies within the cells.

The inventor further transformed the E. coli of the present invention (carrying hfsG, hfsH genes) with hfsA, hfsB and hfsD genes.

They then observed a reduced vacuolization of the cells.

1. A method to obtain a biological glue from a cell, the method comprising the steps of:
selecting a cell lacking of hfsG and hfsH genes;
introducing said hfsG and hfsH genes and into said cell and expressing the corresponding proteins encoded by said hfsG and hfsH genes; and
recovering the glue.

2. The method of claim 1, wherein the cell is a prokaryote cell.

3. The method of claim 2, wherein the cell is E. coli.

4. The method according to claim 1, wherein the cell further comprises one or more gene(s) involved in pigment synthesis.

5. The method of claim 4, wherein the gene(s) involved in pigment synthesis are selected from the group consisting of Carotenoids, Melanin and Violacein.

6. The method according to claim 1, wherein hfsG gene is a gene encoding a glycosyl transferase and having more than 95% sequence identity with SEQ.ID.NO. 2, SEQ.ID.NO.6 and/or SEQ.ID.NO.8.

7. The method according to claim 1, wherein hfsH gene is a gene encoding a polysaccharide deacetylase and having more than 95% sequence identity with SEQ.ID.NO.4, SEQ.ID.NO.10 and/or SEQ.ID.NO.12.

8. The method according to claim 1, wherein the glue is recovered by a drying of the cell culture medium or a drying of the medium conditioned by the cell culture.

9. The method according to claim 1, wherein the glue is recovered by a cell lysis.

10. A genetic construct carrying the genes encoding the HsfG and HsfH proteins and a gene encoding for an antidote protein to a poison protein.

11. The genetic construct of claim 10, wherein the antidote protein is CcdA and the poison protein is CcdB.

12. The genetic construct of claim 10 claim further comprising a gene encoding the HfsA protein.

13. The genetic construct according to claim 10 further comprising genes encoding HfsB protein.

14. The genetic construct according to claim 10 further comprising the gene encoding the HfsC protein.

15. The genetic construct according to claim 10 further comprising the gene encoding the HfsD protein.

16. The genetic construct according to claim 10 further comprising the gene encoding the HfsE protein.

17. An extrachromosomal replicon comprising the genetic construct according to claim 10, being a plasmid.

18. A recombinant cell naturally lacking the hfsG and hfsH genes and comprising the extrachromosomal replicon of claim 17.

19. The cell of claim 18, further comprising a gene encoding a poison protein CcdB present upon the chromosome of the cell.

20. The cell of claim 18 further comprising the deletion of the cps operon.

21. The cell according to claim 18 further comprising the deletion of the cellulose operon.

22. The cell according to claim 18 further comprising the deletion of the PGA operon.

23. The cell according to claim 18, comprising a prokaryote cell.

24. The cell of claim 23 comprising E. coli.

25. A matrix comprising the cell according to claim 18 and a sufficient amount of nutrients for said cell to obtain a biological glue synthesis by said cell.

26. The matrix of claim 25, further comprising elements to form a gel, said elements being selected from the group consisting of an aqueous medium, saccharides and/or proteins.

27. A method for fixing solid elements by a biological glue, the method comprising the step of introducing between the solid elements the matrix of claim 25 for a sufficient time until the nutrients present in the matrix are consumed by the cell leading to death of the cell (lysis), to the release of the produced glue by the cell and to the fixing of the solid elements by the released glue.
